(19) United States
(12) Patent Application Publication
(10) Pub. No.: US 2009/0209647 A1
(43) Pub. Date: Aug. 20, 2009

(54) NUTRITIONAL METHOD

(75) Inventors: Denis Breuille, Lausanne (CH); Sabine Mercier, Montpellier (FR); Isabelle Papet, Clermont-Ferrand (FR); Philippe Patureau Mirand, Clermont-Ferrand (FR); Christiane Obled, Saint Amant-Tallende (FR)

Correspondence Address: Nestle HealthCare Nutrition, 12 Vreeland Road, 2nd Floor, Box 697, Florham Park, NJ, 07932, US

(73) Assignees: NESTEC S.A., Vevey (CH); INSTITUT NATIONAL RECHERCHE AGRONOMIQUE, Paris Cedex (FR)

(21) Appl. No.: 11/994,617

(22) PCT Filed: Jun. 14, 2006
(86) PCT No.: PCT/EP2006/063229

§ 371 Date: Dec. 17, 2008

(30) Foreign Application Priority Data
Jun. 14, 2005 (EP) 05105225.6

(51) Int. Cl. A61K 31/198; A23L 1/305

(52) U.S. Cl. 514/562; 426/656

(57) ABSTRACT

A method of improving nutrition and/or treating low grade inflammation in an elderly human subject comprises administering to said subject a cysteine source so as to provide metabolically available cysteine in the diet of said subject in a proportion relative to all available amino acids which is greater that the proportion of cysteine relative to all amino acids which corresponds to the requirements of a healthy young human subject.

### FIELD OF THE INVENTION

The present invention relates to the improvement of human nutrition. In particular the invention relates to the improvement of nutrition in elderly human subjects.

### BACKGROUND OF THE INVENTION

Ageing is associated with increased levels of inflammatory components in the blood, including acute phase proteins and cytokines. Indeed, modest acute phase protein changes may occur with ageing even among apparently healthy individuals. Thus concentrations of C-reactive protein (CRP), α1-glycoprotein acid or fibrinogen have been found slightly but significantly increased in animals and humans (1-3). Moreover, concentration of the negative acute phase protein, albumin, is decreased (3, 4). Such changes are representative of subclinical inflammation. Indeed, a dysregulation of the immune system occurs in the elderly (5). With respect to cytokines, increased circulating levels of TNF-α and IL-6 have been reported during ageing (6). The activity and cytokine production of blood mononuclear cells is altered with an imbalance between pro- and anti-inflammatory cytokines (7). However, the metabolic and nutritional implications of this low-grade inflammatory state are unclear.

The low-grade inflammation present in the elderly could impact the immune response to additional injury or diseases. An increased risk of death or of developing diseases has been reported in the elderly with elevated levels of cytokines or acute phase proteins (8, 9). Several studies have suggested an altered acute phase response during infection or endotoxemia. Elderly patients with pneumonia exhibited lower cytokine plasma levels and production by peripheral blood monocytes during the acute phase of the infection than young subjects but prolonged inflammatory activity (10,11). Similar results were found in endotoxemia (12).

It is well established that the acute phase response leads to important metabolic changes in general and protein and amino acid metabolism in particular (13-14). Inflammation results in an overall increase of protein metabolism. Increased whole body protein breakdown predominates over the increased whole body protein synthesis leading to a negative protein balance (15, 16). A net catabolism of protein occurs in muscle to provide substrates for synthesis of acute phase proteins or proteins of the immune system (17, 18). During acute diseases, the metabolism of individual amino acids, especially methionine and cysteine, is also altered (19). Methionine is mainly metabolized in the liver through the transmethylation-transsulfuration pathway. The transmethylation pathway leads to homocysteine synthesis. Then homocysteine can be remethylated to form methionine or catabolized via the transsulfuration pathway which ultimately forms cysteine. Under normal circumstances, this pathway constitutes a significant source of cysteine (20, 21). In injury, the contribution of the transsulfuration pathway to the methionine flux increases, suggesting an increased cysteine requirement in diseases (22, 23). Indeed, cysteine is required for the synthesis of taurine and mainly glutathione, which are important compounds for host defense against oxidative stress (13).

In humans, methionine kinetics has been studied in healthy young subjects in relation to the intake of methionine, cysteine or folate and vitamin B₆ (24-27). By contrast, only one study has been devoted to methionine metabolism in the elderly and the influence of inflammation has never been explored in elderly subjects (28).

It is known from U.S. Pat. No. 5,756,481 and U.S. Pat. No. 5,863,906 that nutritional compositions containing a greater proportion of cysteine relative to all amino acids than that which corresponds to the nutritional requirements of a healthy man are useful in the treatment of sepsis or an attack bringing out an inflammatory reaction.

An object of the invention is to improve nutrition in elderly human subjects, in particular elderly human subjects who appear healthy and for example are not suffering from metabolic and/or immune disorders.

### SUMMARY OF THE INVENTION

According to one aspect, the present invention provides a method of improving nutrition in an elderly human subject which comprises administering to said subject a cysteine source so as to provide metabolically available cysteine in the diet of said subject in a proportion relative to all available amino acids which is greater that the proportion of cysteine relative to all amino acids which corresponds to the requirements of a healthy young human subject. Preferably, the method comprises administering from about 2 to about 5 g of cysteine per day.

According to another aspect, the present invention provides a method of treating low grade inflammation in an elderly human subject which comprises administering to a subject suffering from low grade inflammation a therapeutic amount of a nutritional composition which includes a cysteine source in an amount such that the metabolically available cysteine provided to said subject relative to all available amino acids provided by said composition is greater than the proportion of cysteine relative to all amino acids that corresponds to the nutritional requirements of a healthy young human subject. Preferably, the method comprises administering from about 2 to about 5 g of cysteine per day.

According to a further aspect, the present invention provides the use of a cysteine source as a dietary supplement for elderly human subjects.

According to a still further aspect, the present invention provides a method of producing a nutritional composition suitable for administration to elderly human subjects which comprises:

(i) providing a nutritional composition containing amino acids in relative proportions corresponding to the requirements of a healthy young human subject; and
(ii) supplementing said nutritional composition with a cysteine source such that on ingestion by said subject said composition provides metabolically available cysteine in a proportion relative to all available amino acids provided by said composition greater than the proportion of cysteine relative to all amino acids which corresponds to the requirements of a healthy young human subject.

As used herein the term “cysteine source” means any material which provides metabolically available cysteine to the subject and includes in particular free cysteine, a cysteine precursor such as cystathionine, a cysteine prodrug, protein containing cysteine, protein hydrolysates containing cysteine and mixtures thereof.

As used herein the term “elderly human subject” means a human whose body function, for example in terms of metabolism and/or immunological status, has been affected as a result of advancing age. Generally such subjects will have an age of 50 years or more, more particularly 55 years or more, even more particularly 60 years or more, most particularly 65 years or more.

As used herein the term “healthy young human subject” means an adult human whose body function, for example in terms of metabolism and/or immunological status has not been affected as a result of advancing age or by any other pathological condition. Generally such subjects will be aged from 20 years to 40 years, more particularly from 20 to 30 years.

### BRIEF DESCRIPTION OF THE DRAWINGS

FIG. 1 illustrates the protocol of the Study on which the invention is based

FIG. 2 is a schematic description of the methionine cycle with its components

FIG. 3 shows the relative activities of various components of methionine cycle in humans

### DETAILED DESCRIPTION OF THE INVENTION

The present invention is based on a study of methionine kinetics in the elderly compared to young subjects which also explored the effect of ageing on the response to a mild inflammatory challenge induced by a vaccination. More particularly, the aims of the study were to investigate the effects of ageing and mild inflammation on methionine kinetics, especially the bioconversion of methionine into cysteine and the meaning of these metabolic changes in term of sulfur amino acid requirement during ageing.

Methionine is an important amino acid because it is nutritionally indispensable and also the source of sulfur for cysteine synthesis. Cysteine becomes conditionally indispensable in inflammatory conditions (13, 14) and there is evidence of increased prevalence of inflammation with advancing age (1-3, 6). Sulfur amino acid metabolism is regulated through homocysteine production from methionine (transmethylation, TM) and the balance between the two pathways of homocysteine utilization (transsulfuration, TS and remethylation, RM). An understanding of the effect of ageing on these metabolic pathways is essential to improve our knowledge on amino acid requirements in elderly.

The values of methionine fluxes found in the group of young subjects are in the range of those previously reported (20, 21, 36). Methionine-methyl and carboxyl fluxes and the components of the methionine cycle were increased in response to feeding as already shown with similar sulfur amino acid intakes (20). Whatever the nutritional state, methyl- and carboxyl-methionine fluxes, non oxidative methionine disposal and methionine appearance from protein breakdown decreased with ageing. Using a large number of men and women across the adult age span (between 19 and 87 years) and controlled diet and physical exercise, Short et al. (40) have shown that leucine and phenylalanine kinetics decline with age in men and women even after correction for fat-free mass. Up to now, the effect of ageing on methionine cycle was not clear since data on young and old subjects were reported in separate studies (20, 21, 28, 36). Among the components of the methionine cycle, the present study found that only homocysteine remethylation decreased with age despite normal folate status. Indeed, it is well demonstrated that homocysteine remethylation is impaired in folate deficiencies (24). Plasma homocysteine concentration was greater in the old subjects than in the young ones as generally observed (41). In contrast, methionine transmethylation and homocysteine transsulfuration rates were maintained during ageing. However, transsulfuration was better preserved than transmethylation since the ratio TS/TM was greater in elderly than in young subjects. Moreover, the ratio RM/TS and the proportion of the methionine methyl-flux provided by homocysteine remethylation decreased in older people. Taken together, these results indicate for the first time that methionine metabolism was preferentially directed towards transsulfuration and therefore cysteine synthesis in elderly.

Plasma cyst(e)ine concentration was found increased in older subjects as compared to young subjects. During studies of diseases associated with inflammation and oxidative stress, an activation of methionine cycle and transsulfuration pathways allowing an increased cysteine availability for glutathione synthesis (23, 30, 42) was found. Glutathione is the most important intracellular antioxidant of the body and the maintenance of glutathione pools is essential for the defence of the organism (19). In this study, blood glutathione concentration was not modified in the elderly in contrast with previous studies showing a decline of plasma and blood concentrations with ageing (43-44). The concentration of some acute phase proteins, although in the normal range, especially fibrinogen, was found higher in the group of old subjects included in the present study than in the young one. This observation revealed a moderate basal inflammatory state in this group of elderly (66-76 years), healthy subjects. Data obtained in acute inflammation (22, 23, 45) let us hypothesize that the preferential orientation of methionine metabolism towards transsulfuration in old subjects was related to their low-grade inflammatory state.

In the group of young subjects, vaccination, used as a model of moderate inflammatory stress, was also associated with changes in the methionine cycle in favour of a predominance of homocysteine transsulfuration over remethylation with no change in the transmethylation rate. Indeed homocysteine remethylathion was decreased after vaccination in contrast to transsulfuration which was increased, leading to significant variations of the ratios TS/TM and RM/TS. These results are in general agreement with the perturbation of sulfur amino acid metabolism found in acute diseases. The contribution of the transsulfuration pathway to the methionine flux increased in burn patients as compared to controls and an increased cysteine synthesis from methionine has been found in septic rats (22, 23). Moreover, cysteine flux was more stimulated by infection than methionine flux (22). In addition, cysteine catabolism was reduced whereas its utilization for glutathione synthesis was increased (30, 45-47). All these data strongly suggest an increased cysteine utilization even under a mild inflammatory stress.

Methionine balance was significantly decreased after vaccination. Vaccination increased methyl-methionine flux and tended to increase carboxyl-methionine flux and protein turnover in the old subjects. In the same time, transmethylation tended to increase and remethylation to decrease less in old subjects than in the young ones. However, as observed in young people, homocysteine metabolism was oriented in favour of cysteine synthesis after vaccination. This change tended to be less pronounced than in the young subjects. For example, the ratio TS/TM was increased by 21 and 11% by vaccination in the post-absorptive and fed states respectively in young subjects instead of 11 and 8% in the elderly. Taken together, these results may suggest that methionine utilization has to be preserved in elderly subjects after vaccination so that homocysteine remethylation was better maintained than in young subjects. Therefore, the competition between homocysteine remethylation and transsulfuration seems more severe in elderly leading to a trend for a decrease in blood glutathione. Another explanation could be a defective metabolic adaptation to an inflammatory challenge as already established for the immune system with advancing age (5). In the same subjects, we have found lower increases of acute phase proteins in response to vaccination in elderly subjects than in young subjects. There are no other published data on the effect of an inflammatory stress on amino acid metabolism in elderly subjects. It can be hypothesized that the age-related differences in the metabolic response could be linked to alterations of the inflammatory response.

Methionine metabolism was affected after vaccination in agreement with previous data obtained in acute diseases. The preferential methionine metabolism toward cysteine synthesis confirms an increased requirement of sulfur amino acids in these situations. The main finding of this study is a higher proportion of methionine entering the transsulfuration pathway in elderly subjects before vaccination, probably due to a low-grade inflammatory state in these subjects. These data suggest that healthy ageing may be associated with an increased cysteine requirement related to a low-grade inflammatory state. Moreover, the effect of vaccination on methionine kinetics tended to differ in elderly as compared to younger subjects. These findings in term of sulfur amino acid requirement during ageing suggest that improvements in the nutrition of elderly human subjects could be achieved by supplementing the diet of such subjects with sulphur amino acids and in particular cysteine.

Compositions based on amino acids for use according to the invention may be intended to be administered orally, enterally or parenterally. Such compositions contain, in a biologically and nutritionally acceptable medium, a cysteine source, i.e. free cysteine or cysteine in a form in which it is biologically available to the subject such as cysteine precursor, cysteine prodrug, proteins or protein hydrolysates which are rich in cysteine. The compositions contain the cysteine source in a proportion of available cysteine greater than the proportion of cysteine present in a nutritional composition corresponding to the requirements of a healthy young human subject. The proportion of cysteine is determined with respect to all the amino acids present in the composition.

In a preferred composition, cysteine, in available form, is present in a proportion equal to or greater than 3% with respect to all the amino acids present in the composition.

Compositions referred to above may contain the eight essential amino acids, namely leucine, isoleucine, valine, tryptophan, phenylalanine, lysine, methionine and threonine. The compositions may also contain glycine and/or arginine. The compositions can also contain taurine and/or glutamine. The composition may contain all amino acids usually contained in proteins.

The compositions may be provided in a solution form as a mixture of amino acids. In one embodiment, the compositions can optionally be used in the form of pharmaceutically acceptable salts of the amino acids in a medium consisting generally of distilled water. The compositions can, according to one embodiment, contain, per 1 liter of amino acids solutions, the following constituents in the following amounts:

### Leucine 5 to 12 g/l

### Isoleucine 3 to 10 g/l

### Valine 5 to 10 g/l

### Tryptophan 1.0 to 3 g/l

### Phenylalanine 1.5 to 7 g/l

### Lysine 2 to 7 g/l

### Methionine 1.5 to 5 g/l

### Threonine 3.0 to 7 g/l

Cysteine is generally present in this composition in proportions equal to or greater than 3% with respect to the total amount of amino acids present. Preferably, cysteine is present in the composition at a level of from about 3 to about 10% of the total amino acids present.

Cysteine can be used in the form of a cysteine precursor which can be converted to cysteine in vivo, for instance cystathionine. It can be used also as a prodrug or in the form of a pharmaceutically acceptable salt, such as in the L-oxothiazolidinecarboxylic acid form, especially when it is desired to avoid maintaining high cysteine plasma levels. It is, of course, possible to use other cysteine precursors or derivatives which can be converted to cysteine in vivo. Cysteine can be used in a form combined with other amino acids such as in the protein or peptide form. The amounts of prodrug or cysteine precursors, peptide or protein are determined on the basis of available cysteine, i.e. the cysteine which is capable of being released from these derivatives.

It is also possible to use the other amino acids mentioned above in the form of precursors or prodrugs, such as, for example, in the dipeptide form.

The compositions can be provided not only in an aqueous solution form but also in other forms. Thus, cysteine can be administered simply by modifying existing enteral oral formula by introducing therein the amount of cysteine compatible with the proportions in accordance with the invention. Cysteine can also be provided in preparations intended for oral or enteral nutrition, for example by the use of proteins or peptide hydrolysates which are naturally rich in cysteine/cystine.

Cysteine should, in this case, also be present in amounts greater than the proportion of cysteine present in a composition intended for a healthy young human subject, this amount being determined with respect to all amino acids present in the free or combined form. It is also possible to express the necessary amount by taking account of the nitrogen content contained in the cysteine or of these precursors and that of the total amount of nitrogen in the composition. The percentage represents in this case the amount of nitrogen from the cysteine with respect to the total nitrogen present.

Cysteine bonded in a protein or a peptide hydrolysate is preferably present in proportions equal to or greater than 3% with respect to all the amino acids present in the free or bonded form in the composition. When it is expressed as nitrogen content, the amount of nitrogen from free cysteine or cysteine in the form of one of its precursors, prodrug, protein or peptide hydrolysate is greater than or equal to 2.15% with respect to the total nitrogen.

The compositions can be provided in the form of a complete nutritional composition intended for parenteral administration. Such preparations can contain, besides the amino acids or their derivates (peptides), carbohydrate (glucose, fructose, sorbitol, and the like) and/or lipid (fatty acid triglycerides) calorie sources. The lipids can contain long chains, medium chains, or short chains, triglycerides. The composition can also contain electrolytes, trace elements and vitamins. In these nutritional compositions, cysteine or its precursors will be present in proportions greater than 3% with respect to the amount of amino acids present in the nutritive composition.

Compositions intended for parenteral administration can be provided in the form of an aqueous solution or non-aqueous solution, suspension or emulsion.

When the composition is provided in the form of a nutritional composition intended for the oral or enteral route cysteine will be present in proportions greater than 3% with respect to the amount of amino acids present in the nutritive composition. The supplementation of cysteine is obtained either with the amino acid itself, with a prodrug or with proteins or peptide hydrolysates which are particularly rich in cysteine. This composition, besides proteins, amino acids and peptides, can contain carbohydrate (in the form of various hydrochlorides) and/or lipid (triglycerides of fatty acids containing long or medium chains, introduced in the form of oils of various origins) calorie sources, electrolytes, trace elements and vitamins.

Cysteine can also be premixed with the other amino acids which can be used in the compositions for use in accordance with the invention. The cysteine can also be provided in the form of an aseptic powder which can be rehydrated at the time of administration or can be stored in the form of a frozen or refrigerated concentrate which is defrosted and mixed to the suitable concentration at the time of use.

These compositions can be administered by devices known in the methods of oral, parenteral or enteral administration.

A preferred dose of cysteine is from about 2 g to about 5 g per day. The dose may be administered as a single dose or as multiple sub-doses, e.g. if the efficacious dose is 3 g per day, the dose may be two 1.5 g sub-doses administered per day, or three 1 g sub-doses administered per day.

Further details of cysteine containing compositions can be found in U.S. Pat. Nos. 5,756,481 and 5,863,906, the contents of which are hereby incorporated by reference.

Experimental details of the study on which the present invention is based are as follows:

### Subjects and Methods

### Subjects and Protocol

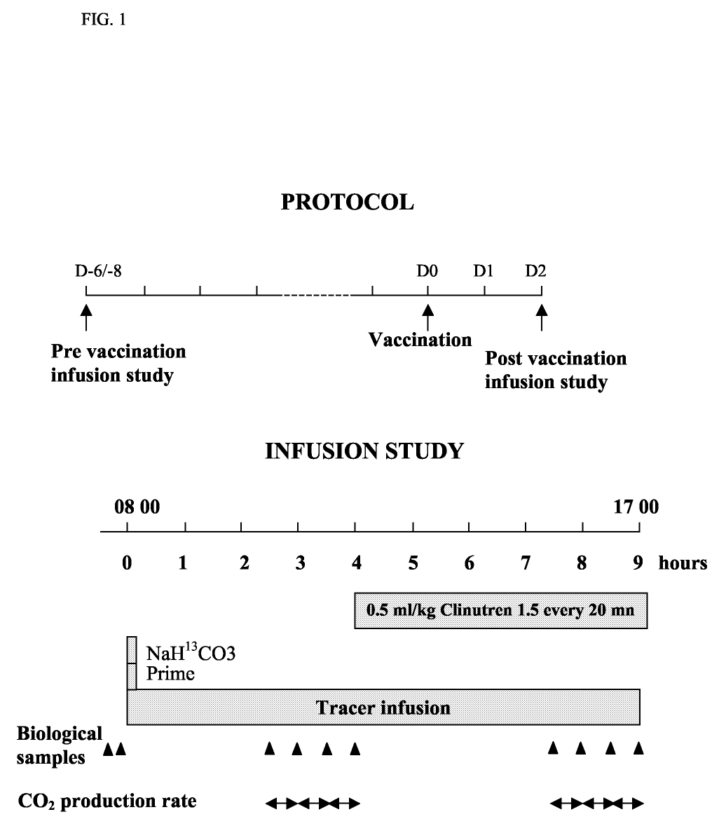

Seven elderly volunteers (3 women and 4 men) aged 66 to 76 years were compared with 8 young volunteers (4 women and 4 men) aged 22 to 26 years (Table 1). Volunteers gave their informed consent to participate in the protocol, which was approved by the local ethical committee for biomedical research (CCPRB Auvergne). Volunteers were studied at 2 time points, 6-8 days before vaccination and 2 days after vaccination (FIG. 1). Vaccination was performed by intramuscular injection of DT-Polio (diphtheria, tetanus, poliomyelitis) and Typhim Vi (typhoid) vaccines (Institut Merieux, Lyon, France).

Examples of menus were furnished to each volunteer in order to standardize their diet to provide adequate energy intake on the basis of their estimated energy expenditure and adequate protein intake for 4 days before each infusion study. On the evening before each infusion study, the subjects took their meal in the Human Nutrition Unit (Clermont-Ferrand). At 0700 on the infusion day, an intravenous catheter was placed in a forearm vein for tracer infusion and in a dorsal vein of the hand for arterialised blood sampling after introduction of the hand into a ventilated box heated to 60° C. At 0800, a priming dose of sodium [¹³C] bicarbonate (0.1 mg/kg) (Eurisotop, Saint Aubin, France), L-[1-¹³C, methyl-²H₃] methionine (2.5 μmol/kg, Cambridge Isotope Laboratory, Andover, Mass., USA) was administered intravenously, and an infusion of L-[1-¹³C, methyl-2H₃] methionine was begun and continued for 9 h (2.5 μmol/kg.h). After the first 4 h, subjects were given small meals every 20 min for 5 h (FIG. 1). The diet given as a drink (Clinutren 1.5, 1.5 ml/kg.h, Nestle, France) provided five-twelfth the total daily protein and energy intake (1 g/kg.d and 27 kcal/kg.d). The methionine and cyst(e)ine supply was 25 and 7.8 mg/kg.d respectively. Blood and breath samples were taken just before the start and at half-hourly intervals during the last 90 min of each metabolic phase (post absorptive and fed states). Blood was collected in heparin and EDTA-containing tubes. After centrifugation, plasma was stored at −80° C. until analysed. Breath samples were placed in evacuated tubes and stored at room temperature until measurements of ¹³CO₂ in the expired air by isotope ratio mass spectrometry. CO₂ production was determined in the fasted and fed states by indirect calorimetry (Deltatrac, Datex, Geneva, Switzerland).

To determine whether the experimental diet alters breath ¹³CO₂ baseline enrichment during the fed state, 6 additional subjects were studied in the same conditions than in the experiment except than no infusion of isotope was given. The subjects drank Clinutren and breath samples were analysed for ¹³CO₂ enrichment. Data for these six subjects were averaged for the last 90 min of the fed period and this value was applied to the carbon-13 enrichment determined for each half-hourly period during the fed state.

### Analytical Methods

The free amino acids were isolated from a 1 ml plasma sample by acid precipitation of protein. 50 μl of β-mercaptoethanol was added to the sample in order to preserve methionine. Plasma enrichment of free methionine was measured by using a tert-butyldimethylsilyl derivative and gas chromatography-mass spectrometry under electron impact ionization (Automass, Thermo Quest Finnigan, Paris, France). Methionine, [1-¹³C] methionine and [1-¹³C, methyl-2H₃] methionine were monitored at a mass-to-charge ratio (m/z) of 320, 321 and 324 respectively. Calibration graphs were prepared from standard mixtures of either [1-¹³C] methionine or [1-¹³C, methyl-²H₃] methionine. ¹³CO₂ enrichment was measured by gas chromatography isotope ratio mass spectrometry (Microgas, Micromass, Manchester, UK).

Total, free and bound cysteine were measured in plasma according to the method of Malloy et al. (29). Briefly, total free cysteine was measured on plasma treated with dithiothreitol before deproteinization. Total free cysteine (free cysteine and cystine) was determined on plasma treated with dithiothreitol after deproteinization. Free cysteine was measured on deproteinized plasma without any reducing treatment. Cystine was then calculated by difference between unbound cysteine and free cysteine. Total erythrocyte glutathione was measured by a standard enzymatic recycling procedure as described previously (30). Plasma total homocysteine was measured as described by Pfeifer et al. (31) and plasma folates as described by Wright et al (32).

### Experimental Model

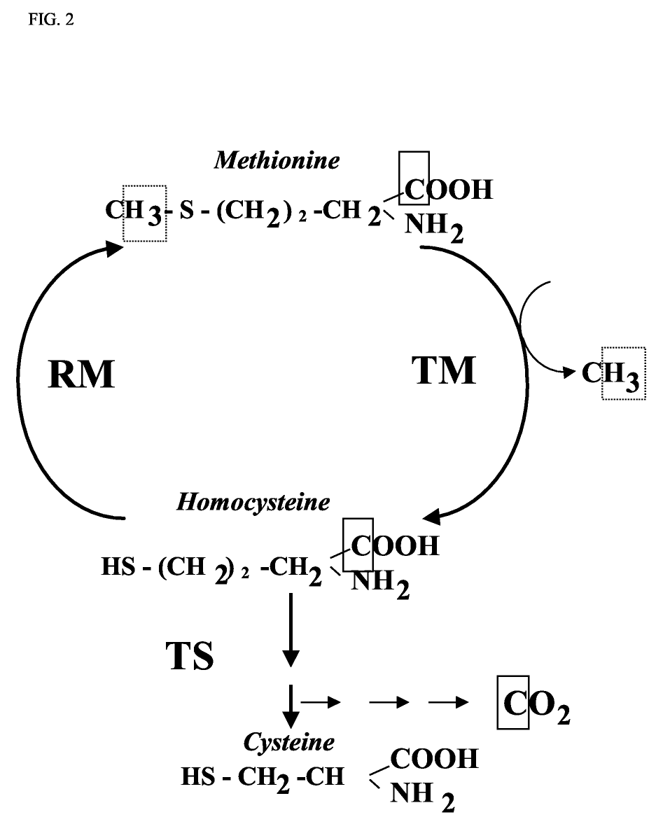

Methionine kinetics were calculated according to the model of Storch et al. (20) and Raguso et al (33) (FIG. 2). Briefly, whole-body methionine-methyl flux rate (Qₘ) and whole body methionine-carboxyl flux rate (Q_{c}) were calculated as follows

Qₘ=(I×Eᵢ)/(E₄×R)

Q_{c}=(I×Eᵢ)/(E₁+E₄×R)

where I and Eᵢ are the infusion rate and the isotope enrichment respectively of [1-¹³C, methyl-²H₃] methionine, and E₁ and E₄ are the plateau plasma enrichments of [1-¹³C] methionine (m+1) and [1-¹³C, methyl-2H₃] methionine (m+4) respectively. The correction factor R was used for the plasma intracellular gradient in methionine enrichment. The value used was 0.8 according to Storch et al. (20).

In steady state conditions, the flux is the sum of inputs or the sum of outputs. Hence in the post absorptive state

Q_{c}=Bₘₑₜ+I=Sₘₑₜ+TS

and in the fed state

Q_{c}=Bₘₑₜ+A=Sₘₑₜ+TS

where Bₘₑₜ is the rate of methionine appearance from protein breakdown, Sₘₑₜ is the rate of methionine disappearance via non oxidative metabolism, an index of the rate of protein synthesis, TS is the transsulfuration rate and A is the total methionine entry from the tracer and the alimentary input.

In steady state conditions, whole-body methionine-methyl flux rate can also be related to its individual components as follows

Qₘ=I(or A)+Bₘₑₜ+RM=Sₘₑₜ+TM

where RM is the remethylation rate and TM, the transmethylation rate.

Therefore, RM=Qₘ−Q_{c} and TM=TS+RM.

TS was calculated as follows

TS=V¹³CO₂/(E₁+E₄×R)

where V¹³CO₂ is the rate of ¹³C output in expired air corrected for the retention of ¹³CO₂ according to Hoerr et al. (34).

Finally Sₘₑₜ is calculated from the difference between Q_{c} and TS, Bₘₑₜ from the difference between Q_{c} and I or A, and methionine balance from the difference between Sₘₑₜ and Bₘₑₜ.

### Statistical Methods and Data Evaluation

Differences between young and old subjects were tested by unpaired t-test. For data obtained only in the basal state (post-absorptive state before beginning tracer infusion), differences were tested by ANOVA for repeated measurements (Statview) for the effect of age and vaccination. Otherwise, the effects of age, vaccination and nutritional state (post-absorptive or fed) were analysed by using a repeated measure analysis of variance (with age as the between-subject factor and vaccination and nutritional state as the within-subject factors). Differences were considered to be significant when P<0.05.

### Results

### Subjects

The elderly subjects included in the study were stringently selected for good health and clinical and biological features matched the admission criteria of the SENIEUR protocol (35). However, these subjects showed greater plasma concentrations of some acute phase proteins, such as α1-acid glycoprotein and fibrinogen and tended to show higher concentrations of CRP (P=0.077) than did the young subjects, suggesting a low grade inflammatory state. In contrast, the plasma concentration of folates was similar in the two groups (Table 1).

There was no significant effect of age or vaccination on plasma methionine and erythrocyte glutathione concentrations. Vaccination had no effect on plasma cysteine and homocysteine concentrations. In contrast, the plasma concentration of the various forms of cysteine and of total homocysteine were greater in elderly than in young subjects (Table 2).

### Methionine Fluxes

The isotopic enrichments of plasma methionine and of ¹³C in expired air during the fasting and fed periods before and after vaccination are summarized in Table 3 for each group.

Whatever age and treatment, there was a significant effect of the nutritional state on methionine fluxes that were generally increased in the fed state except methionine released from protein breakdown which was decreased (P<0.001) (Table 4). Before vaccination, methionine methyl flux was greater in young than in elderly subjects. There was a significant interaction between age and vaccination (P=0.027), indicating that the effects of vaccination on methionine-methyl flux differed in young and old people. Indeed, no difference between the two groups was observed after vaccination. For the other methionine fluxes, there were no significant interactions between age, vaccination and nutritional state, but there were significant main effects (Table 4). Without regard to vaccination and nutritional state, methionine-carboxyl flux (P=0.036), methionine non oxidative disposal (P=0.03) and methionine endogenous fluxes (P=0.033) were lower in elderly than in young subjects. A similar trend (P=0.077) was observed for methionine transmethylation. Whatever the age of the subject and the nutritional state, vaccination significantly increased transsulfuration (5.18±0.17 vs 5.73±0.17 μmol/kg.h, P=0.035) and reduced methionine balance (4.30±0.17 vs 3.68±0.17 μmol/kg.h, P=0.022). Homocysteine remethylation was significantly reduced by age (5.33±0.27 vs 4.00±0.29 μmol/kg.h, P=0.006) and vaccination (5.00±0.14 vs 4.44±0.14 μmol/kg.h, P=0.022). The interaction age-vaccination tended to be significant for RM which tended to be less decreased by vaccination in elderly, and Sₘₑₜ and Bₘₑₜ which tended to increase after vaccination in elderly.

### Relative Activities of Various Components of Methionine Cycle

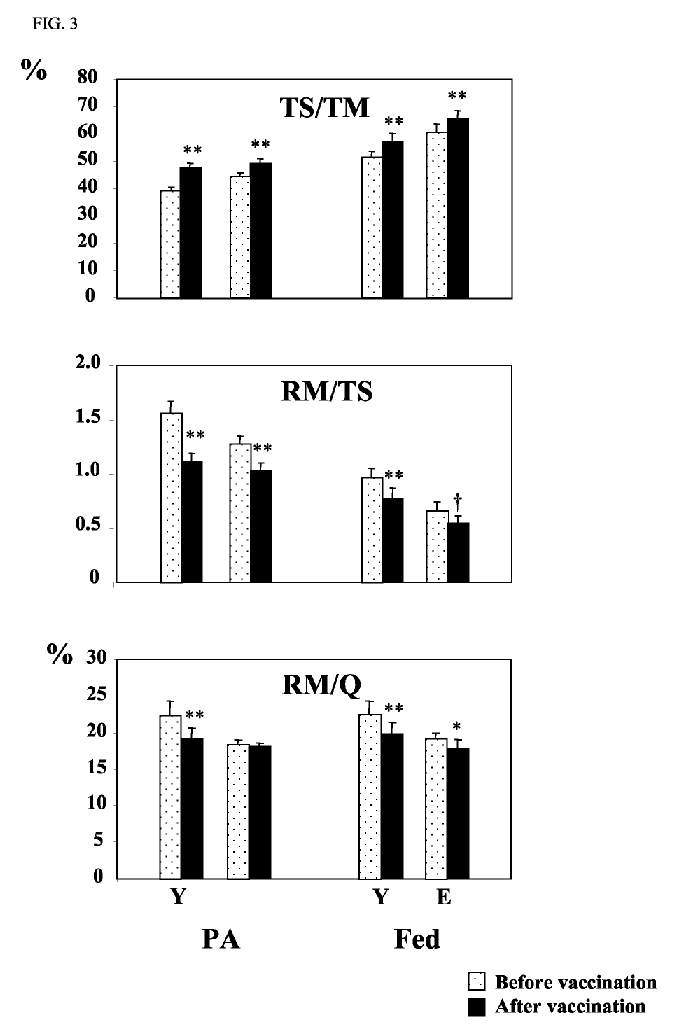

The proportion of methionine transmethylation that entered transsulfuration (TS/TM) was significantly increased by age (P=0.024), vaccination (P=0.0006) and nutritional state (P=0.0001) without any interaction between age, vaccination and nutritional state (FIG. 3). The ratio of remethylation to transsulfuration (RM/TS) was decreased by all factors (age: P=0.035, vaccination: P=0.0005, and nutritional state: P=0.0001). A significant interaction was found between vaccination and nutritional state (P=0.006), indicating that the effect of vaccination was less pronounced in the fed state than in the post absorptive (PA) state. The proportion of methionine-methyl flux provided by homocysteine remethylation (RM/Qm) was significantly reduced by age (P=0.013) and vaccination (P=0.013) but there was no interaction between age, vaccination and nutritional state.

The tables referred to above are as follows:

TABLE 1
Subject characteristics¹

| | Young | Elderly |
|---|---|---|
| Age (y) | 23 ± 1 | 70 ± 1 |
| Weight (kg) | 67.3 ± 3.1 | 69.2 ± 2.2 |
| Height (m) | 1.73 ± 0.04 | 1.62 ± 0.03 |
| BMI (kg/m²) | 22.3 ± 0.4 | 26.3 ± 0.5 |
| Plasma folates (nmol/L) | 13.5 ± 2.1 | 11.3 ± 2.9 |

¹ X̅ ± SE

TABLE 2
Plasma concentrations of methionine, cysteine and homocysteine and
erythrocyte glutathione concentration in the post-absorptive state¹

| | Young | | Elderly | |
|---|---|---|---|---|
| | Before vacc. | After vacc. | Before vacc. | After vacc. |
| Methionine (μmol/L) | 15.4 ± 1.0 | 15.6 ± 0.9 | 14.4 ± 2.0 | 15.2 ± 0.7 |
| Total cysteine² (μmol/L) | 225 ± 5 | 219 ± 6 | 269 ± 10 | 266 ± 8 |
| Total free cysteine² (μmol/L) | 130 ± 3 | 127 ± 4 | 158 ± 7 | 158 ± 6 |
| Free cystine² (μmol/L) | 51 ± 2 | 48 ± 3 | 61 ± 3 | 60 ± 3 |
| Free cysteine² (μmol/L) | 29 ± 1 | 31 ± 2 | 37 ± 2 | 37 ± 2 |
| Total homocysteine³ (μmol/L) | 6.7 ± 0.7 | 6.6 ± 1.0 | 10.2 ± 0.9 | 9.0 ± 0.6 |
| Erythrocyte glutathione (mmol/L) | 2.04 ± 0.10 | 2.07 ± 0.11 | 2.03 ± 0.10 | 1.83 ± 0.14 |

¹ X̅ ± SE
²Age P < 0.01, Vaccination NS, Age × Vaccination NS
³Age P < 0.05, Vaccination NS, Age × Vaccination

TABLE 3
Plasma isotope enrichments, ¹³CO₂ enrichment and carbon dioxide production in young and elderly subjects before and after vaccination¹

| | Young | | | | Elderly | | | |
|---|---|---|---|---|---|---|---|---|
| | Before vaccination | | After vaccination | | Before vaccination | | After vaccination | |
| | Fasted | Fed | Fasted | Fed | Fasted | Fed | Fasted | Fed |
| [1-¹³C, methyl-²H₃]methionine | 12.0 ± 0.5 | 9.2 ± 0.5 | 12.1 ± 0.5 | 9.5 ± 0.5 | 15.2 ± 0.5 | 10.8 ± 0.3 | 14.0 ± 0.5 | 10.3 ± 0.5 |
| (MPE) | | | | | | | | |
| [1-¹³C]methionine (MPE) | 2.52 ± 0.11 | 1.98 ± 0.13 | 2.16 ± 0.11 | 1.76 ± 0.14 | 2.54 ± 0.16 | 2.05 ± 0.19 | 2.18 ± 0.11 | 1.59 ± 0.19 |
| Breath ¹³CO₂ enrichment | 3.8 ± 0.3 | 6.2 ± 0.5 | 4.3 ± 0.2 | 6.3 ± 0.4 | 4.8 ± 0.1 | 8.4 ± 0.7 | 4.8 ± 0.3 | 8.2 ± 0.7 |
| (MPE × 10³) | | | | | | | | |
| Carbon dioxide production | 185 ± 10 | 224 ± 10 | 190 ± 8 | 235 ± 11 | 156 ± 8 | 212 ± 12 | 163 ± 11 | 215 ± 11 |
| (ml/min) | | | | | | | | |

¹ X̅ ± SE

TABLE 4
Methionine fluxes in young and elderly subjects before and after vaccination¹

| | Fluxes (μmol/kg · h) | | | | | | | |
|---|---|---|---|---|---|---|---|---|
| | Young | | | | | Elderly | | |
| | Before vaccination | | After vaccination | | Before vaccination | | After vaccination | |
| | Fasted | Fed | Fasted | Fed | Fasted | Fed | Fasted | Fed |
| Qm methionine² | 24.9 ± 1.1 | 32.6 ± 1.8 | 24.3 ± 1.1 | 31.1 ± 1.6 | 19.7 ± 0.7 | 27.7 ± 0.8 | 21.2 ± 0.6 | 29.0 ± 1.3 |
| Qc methionine³ | 19.2 ± 0.5 | 25.2 ± 1.1 | 19.5 ± 0.7 | 24.9 ± 1.2 | 16.1 ± 0.6 | 22.4 ± 0.5 | 17.3 ± 0.6 | 23.8 ± 1.3 |
| TM⁴ | 8.2 ± 0.6 | 13.5 ± 0.8 | 8.0 ± 0.3 | 13.4 ± 1.0 | 6.0 ± 0.3 | 12.5 ± 0.4 | 6.9 ± 0.4 | 12.8 ± 0.6 |
| TS⁵ | 3.3 ± 0.2 | 7.0 ± 0.5 | 3.9 ± 0.2 | 7.7 ± 0.6 | 2.7 ± 0.2 | 7.7 ± 0.5 | 3.2 ± 0.3 | 8.2 ± 0.6 |
| RM⁶ | 5.0 ± 0.4 | 6.5 ± 0.4 | 4.2 ± 0.2 | 5.6 ± 0.4 | 3.3 ± 0.1 | 4.9 ± 0.3 | 3.5 ± 0.2 | 4.3 ± 0.3 |
| S³ | 16.0 ± 0.5 | 18.0 ± 1.3 | 15.5 ± 0.7 | 17.1 ± 1.1 | 13.5 ± 0.5 | 14.7 ± 0.9 | 14.3 ± 0.3 | 15.3 ± 0.9 |
| B³ | 16.8 ± 0.6 | 8.5 ± 1.2 | 17.0 ± 0.7 | 8.3 ± 1.3 | 13.8 ± 0.6 | 5.8 ± 0.6 | 15.3 ± 0.5 | 7.2 ± 1.1 |
| Balance⁵ | −0.9 ± 0.2 | 9.5 ± 0.5 | −1.5 ± 0.2 | 8.8 ± 0.7 | −0.3 ± 0.2 | 8.8 ± 1.3 | −0.8 ± 0.3 | 8.3 ± 1.2 |

¹ X̅ ± SE
²There was a significant effect of age and nutritional state, and a significant interaction between age and vaccination
³There was a significant effect of age and nutritional state, interaction between age and vaccination P = 0.20, 0.11 and 0.14 for Qc, NOLD and B respectively
⁴There was a significant effect of nutritional state, interaction between age and vaccination P = 0.25
⁵There was a significant effect of vaccination and nutritional state
⁶There was a significant effect of age, vaccination and nutritional state, interaction between age and vaccination P = 0.12

A more detailed explanation of the figures referred to above is as follows:

FIG. 1. Study protocol

FIG. 2. A schematic description of the methionine cycle with its components: transmethylation (TM), remethylation (RM) and transsulfuration (TS). If methionine is labelled on the methyl group and the carboxyl group, the methyl label will be lost during transmethylation and homocysteine remethylation will produce methionine labelled only on the carboxyl group. The carboxyl label will be lost during transsulfuration and will appear in carbon dioxide in breath.

FIG. 3. Relative activities of various components of methionine cycle in humans Data are shown as means±SEM.

TS/TM: main effects of age (P<0.05), vaccination (P<0.001) and nutritional state (P<0.001). RM/TS: main effects of age (P<0.05), vaccination (P<0.001) and nutritional state (P<0.001), vaccination by nutritional state interaction (P<0.01), age by vaccination interaction (P=0.19). RM/Qm: main effects of age (P<0.05) and vaccination (P<0.05). Significantly different from before vaccination ** P<0.01; * P<0.05; † P=0.077.

Y=young subjects; E=elderly subjects.
PA=post absorptive state.

### REFERENCES

1. Ballou S P, Lozanski F B, Hodder S, et al. Quantitative and qualitative alterations of acute-phase proteins in healthy elderly persons. Age and Ageing 1996; 25: 224-30.
2. Fu A, Nair K S. Age effect on fibrinogen and albumin synthesis in humans. Am J Physiol 1998; 275:E1023-30.
3. Papet I, Dardevet D, Somet C, et al. Acute phase protein levels and thymus, spleen and plasma protein synthesis rates differ in adult and old rats. J Nutr 2003; 133:215-9.
4. Salive M E, Cornoni-Huntley, J., Phillips, C. L., et al. Serum albumin in older persons: relationship with age and health status. J. Clin. Epidemiol. 1992; 45:213-21.
5. Bruunsgaard H, Pedersen M, Pedersen B K. Aging and proinflammatory cytokines. Cur Opinion Hematol 2001; 8:131-6.
6. Forsey R J, Thompson J M, Ernerudh J, et al. Plasma cytokine profiles in elderly humans. Mech Ageing Dev. 2003; 124:487-93.
7. Saurwein-Teissl M, Blasko I, Zisterer K, Neuman B, Lang B, Grubeck-Loebenstein B. An imbalance between pro- and anti-inflammatory cytokines, a characteristic feature of old age. Cytokine. 2000; 12:1160-1.
8. Ballou S P, Kushner, I. Chronic inflammation in older people: recognition, consequences, and potential intervention. Clinics in Geriatric Medicine 1997; 13:653-69.
9. Harris T B, Ferrucci L, Tracy R P, et al. Associations of elevated interleukin-6 and C-reactive protein levels with mortality in the elderly. Am J. Med. 1999; 106:506-12.
10. Bruunsgaard H, Skinhoj P, Qvist J, Pedersern B K. Elderly human show prolonged in vivo inflammatory activity during pneumococcal infections. J Infect Dis 1999; 180: 551-4.
11. Gon Y, Hashimoto S, Hayashi S, Koura, T, Matsumoto, K, Horie T. Lower serum concentrations of cytokines in elderly patients with pneumonia and the impaired production of cytokines by peripheral blood monocytes in the elderly. Clin Exp Immunol 1996; 106:120-6.
12. Krabbe Kans., Bruunsgaard H, Hansen C M, et al. Ageing is associated with a prolonged fever response in human endotoxemia. Clin Diagn Lab Immunol. 2001; 8:333-8.
13. Obled C, Papet I, Breuillé D. Metabolic bases of amino acid requirements in acute diseases. Cur Opinion Clin Nutr Metabol Care 2002; 5:189-97.
14. Reeds P J, Jahoor F. The amino acid requirements of disease. Clin Nutr 2001; 20:15-22.
15. Mansoor O, Beaufrère B, Boirie Y, et al. Increased mRNA levels for components of the lysosomal, Ca2+-activated and ATP-ubiquitin-dependent proteolytic pathways in skeletal muscle from head trauma patients. Proc Natl Acad of Sci USA 1996; 93:2714-8.
16. Mansoor O, Cayol M, Gachon P, et al. Albumin and fibrinogen synthèses increase while muscle protein synthesis decreases in head-injured patients. Am J Physiol 1997; 273: E898-902.
17. Papet I, Ruot B, Breullé D, et al. Bacterial infection affects protein synthesis in primary lymphoid tissues and circulating lymphocytes of rats. J Nutr 2002; 132:2028-32.
18. Ruot B, Breuillé D, Rambourdin F, Bayle G, Capitan P, Obled C. Synthesis rate of plasma albumin is a good indicator of liver albumin synthesis in sepsis. Am J Physiol 2000; 279:E244-51.
19. Obled C, Papet I, Breuillé D. Sulfur-containing amino acids and glutathione in diseases. In: Cynober L A, ed. Metabolic and Therapeutic Aspects of Amino Acids in Clinical Nutrition. CRC Press 2004:667-87.
20. Storch K J, Wagner D A, Burke J F, Young V R. Quantitative study in vivo of methionine cycle in humans using [methyl-²H₃]- and [1-¹³C]methionine. Am J
21. Storch K J, Wagner D A, Burke J F, Young V R. [1-13C, methyl-2H3] methionine kinetics in humans: methionine conservation and cystine sparing. Am J Physiol 1990; 258: E790-8.
22. Malmezat T, Breuillé D, Pouyet C, et al. Methionine transsulfuration is increased during sepsis in rats. Am J Physiol 2000; 279:E1391-7.
23. Yu Y M, Burke J F, Young V R. A kinetic study of L-2H3-methyl-1-13C-methionine in patients with severe burn injury. J Trauma 1993; 35: 1-7.
24. Cuskelly G J, Stacpoole P W, Williamson J, Baumgartner T G, Gregory J F. Deficiencies of folate and vitamin B-6 exert distinct effects on homocysteine, serine, and methionine kinetics. Am J Physiol 2001; 281:E1182-90.
25. Davis S R, Stacpoole P W, Williamson J, et al. Tracer-derived total and folate-dependent homocysteine remethylation and synthesis rates in humans indicate that serine is the main one-carbon donor. Am J Physiol 2004; 286:E272-9.
26. Di Buono M, Wykes L J, Cole D E, Ball R O, Pencharz P B. Regulation of sulfur amino acid metabolism in men in response to changes in sulfur amino acid intakes. J Nutr 2003; 133:733-9.
27. Hiramatsu T, Fukagawa N K, Marchini J S, et al. Methionine and cysteine kinetics at different intakes of cystine in healthy adult men. Am J Clin Nutr 1994; 60:525-33.
28. Fukagawa N K, Yu Y M, Young V R. Methionine and cysteine kinetics at different intakes of methionine and cystine in elderly men and women. Am J Clin Nutr 1998; 68:380-8.
29. Malloy M H, Rassin D K, Gaull G E. A method for measurement of free and bound plasma cyst(e)ine. Anal Biochem 1981; 113:407-15.
30. Malmezat T, Breuillé D, Capitan P, Patureau Mirand P, Obled C. Glutathione turnover is increased during the acute phase of sepsis in rats. J Nutr 2000; 130:1239-46.
31. Pfeiffer C M, Huff D L, Gunter E W. Rapid and accurate HPLC assay for plasma total homocysteine and cysteine in a clinical laboratory setting. Clin Chem. 1999; 45:290-2.
32. Wright A J, Finglas P M, Dainty J R, et al. Single oral doses of 13C forms of pteroylmonoglutamic acid and 5-formyltetrahydrofolic acid elicit differences in short-term kinetics of labelled and unlabelled folates in plasma: potential problems in interpretation of folate bioavailability studies. Br J Nutr 2003; 90:363-371.
33. Raguso C A, Regan M M, Young V R. Cysteine kinetics and oxidation at different intakes of methionine and cystine in young adults. Am J Clin Nutr 2000; 71:491-9.
34. Hoerr R A, Yu Y M, Wagner D A, Burke J F, Young V R. Recovery of 13C in breath from NaH13CO₂ infused by gut and vein: effect of feeding. Am J Physiol 1989; 257:E426-38.
35. Ligthart G J, Corberand J X, Fournier C, et al. Admission criteria for immunogerontological studies in man: the SENIEUR protocol. Mech Ageing Dev 1984; 28:47-55.
36. Fukagawa N K, Ajami A M, Young V R. Plasma methionine and cysteine kinetics in response to an intravenous glutathione infusion in adult humans. Am J Physiol 1996; 270:E20914.
37. Arnal M A, Mosoni L, Boirie Y, et al. Protein turnover modifications induced by the protein feeding pattern still persist after the end of the diets. Am J Physiol 2000; 278:E902-9.
38. Fereday A, Gibson N R, Cox M, Pacy P J, Millward D J. Protein requirements and ageing: metabolic demand and efficiency of utilization. Brit J Nutr 1997; 77:685-702.
39. Welle S, Thornton C, Statt M, McHenry B. Postprandial myofibrillar and whole body protein synthesis in young and old human subjects. Am J Physiol 1994; 267:E599-E604.
40. Short K R, Vittone J, Bigelow M L, Proctor D N, Nair Kans. Age and aerobic exercise training effects on whole body and muscle protein metabolism. Am J Physiol 2004; 286: E92-E101.
41. Powers R W, Majors A K, Lykins D L, Sims C J, Lain K Y, Roberts J M. Plasma homocysteine and malondialdehyde are correlated in an age- and gender-specific manner. Metabolism Clin Experimental 2002; 51: 1433-8.
42. Mosharov E, Cranford M R, Banerjee R. The quantitatively important relationship between homocysteine metabolism and glutathione synthesis by the transsulfuration pathway and its regulation by redox changes. Biochem 2000; 39:13005-11.
43. Hemanz A, Fernandez-Vivancos E, Montiel C, Vazquez J J, Arnalich F. Changes in the intracellular homocysteine and glutathione content associated with aging. Life Sci 2000; 67:1317-24.
44. Lang Calif., Naryshkin S, Schneider D L, Mills B J, Lindeman R D. Low blood glutathione levels in healthy aging adults. J Lab Clin Med 1992; 120:720-5.
45. Breuillé D, Mansoor O, Buffière C, et al. Cysteine requirement is increased in ICU patients. Clin Nutr 2001; 20:43.
46. Hunter E A L, Grimble R F. Dietary sulphur amino acid adequacy influences glutathione synthesis and glutathione dependent enzymes during the inflammatory response to endotoxin and tumour necrosis factor-alpha in rats. Clin Sci 1997; 92: 297-305.
47. Malmezat T, Breuillé D, Pouyet C, Patureau Mirand P, Obled C. Metabolism of cysteine is modified during the acute phase of sepsis in rats. J Nutr 1998; 128: 97-105.
48. Young V R, Wagner D A, Burini R, Storch K J. Methionine kinetics and balance at the 1985/FAO/WHO/UNU intake requirement in adult men studied with L-[2H3-methyl-1-13C] methionine as a tracer. Am J Clin Nutr 1991; 54:377-85.

1. A method of improving nutrition in an elderly human comprising administering to the human a diet comprising a cysteine source so as to provide that provides metabolically available cysteine in the diet of said to the human in a proportion relative to all available amino acids which is greater than a proportion of cysteine relative to all amino acids which corresponds to the requirements of a healthy young human subject.

2. A method of treating low grade inflammation in an elderly human comprising administering to an elderly human suffering from low grade inflammation a therapeutic amount of a nutritional composition which comprises a cysteine source in an amount such that metabolically available cysteine provided to the human relative to all available amino acids provided by the nutritional composition is greater than a proportion of cysteine relative to all amino acids that corresponds to the nutritional requirements of a healthy young human subject.

3. A method of providing a dietary supplement for an elderly human comprising the steps of using a source of cysteine.

4. A method of producing a nutritional composition suitable for administration to an elderly human comprising:
providing a nutritional composition containing amino acids in relative proportions corresponding to the requirements of a healthy young human subject; and
supplementing the nutritional composition with a cysteine source such that on ingestion by the elderly human the composition provides metabolically available cysteine in a proportion relative to all available amino acids provided by the composition greater than the proportion of cysteine relative to all amino acids which corresponds to the requirements of a healthy young human subject.
